(19) United States
(12) Patent Application Publication
(10) Pub. No.: US 2005/0238719 A1
(43) Pub. Date: Oct. 27, 2005

(54) Film coating

(75) Inventor: Ferenc Buzsaky, Sodertalje (SE)

Correspondence Address: WHITE & CASE LLP;PATENT DEPARTMENT, 1155 AVENUE OF THE AMERICAS, NEW YORK, NY, 10036, US

(73) Assignee: AstraZeneca AB, Sodertalje (SE)

(21) Appl. No.: 10/523,553

(22) PCT Filed: Jul. 28, 2003
(86) PCT No.: PCT/GB03/03195

§ 371 Date: Jan. 31, 2005

(30) Foreign Application Priority Data
Aug. 1, 2002 (SE) 0202353-9

(51) Int. Cl. A61K 9/24

(52) U.S. Cl. 424/472

(57) ABSTRACT

A film coating composition suitable for use in coating pharmaceutical formulations comprising: a) an acrylic polymer, which is Eudragit® NE30D, b) an anti-sticking agent, which is glyceryl monostearate (GMS), c) a surface active agent wherein the surface active agent is in an amount less than 1.3% by weight of the dispersion, and d) a water-containing liquid, wherein the dispersion does not contain a vinyl acetate polymer.

### FIELD OF THE INVENTION

The present invention relates to a new film coating. More specifically the present invention relates to a new film coating for the achievement of modified release from pharmaceutical formulations such as tablets, pellets, etc., wherein the film coating may be applied in a substantially aqueous environment. Furthermore, the invention provides a process for the preparation of such a film coating.

### BACKGROUND OF THE INVENTION

Oral administration of a drug is the most convenient for the patient. Oral formulations must also meet the requirements of safety and simplicity. Depending on the drug properties and the therapeutic requirements, different approaches are taken during its formulation to obtain a suitable drug delivery profile. For example, a sparingly soluble drug which is given once a day requires a different type of formulation than an easily soluble drug which is taken several times a day.

Different formulations can have different mechanisms for controlling the release of the active drug. In the thesis by Sandberg 1994, extended-release (ER) formulations of different types of drugs were reviewed. It was concluded that in principle two types of ER dosage forms exist: (i) the matrix system where the drug is mixed with the matrix material (often a polymer or a wax) and (ii) the drug reservoir system where the drug is formulated into a core (tablet or pellets) surrounded by a polymeric film. The film coating is then a release rate-controlling barrier determined by many factors including its dissolution rate, its permeability, the solubility of the substance, etc.

From a flexibility point of view the formulation of a drug into small discrete units coated with a film has gained much intention. Such a formulation shows several interesting features such as flexibility in dosage and release properties. In addition, tablets made from these discrete units are easily divisable. In a numbers of studies it was shown that safe, simple and convenient therapy can be achieved utilizing this principle for the (water soluble) drug metoprolol and its salts (Ragnarsson et al, Drug Develop Ind Pharmacy 13, 1495 (1987); Sandberg et al, Eur 3 Clin Pharmacol 33, S3 (1988) and S9 (1988); Ragnarsson et al, Int J Pharmaceuticl 79, 223 (1992); Sandberg et al, Ibid 68, 167 (1991); Sandberg et al, Pharmaceuticl Res 10, 28 (1993); Sandberg et al, Drug Invest 6, 320 (1993); Sandberg, Thesis Uppsala University, (1994).

The formulation of metoprolol into pellets according to the above mentioned references utilized a film coating sprayed from a solution containing a mixture of different celluloses in an organic solvent. However, for environmental reasons it would be preferable to use water based film-forming systems for this and other drugs to be formulated as pellets systems. Thus, much effort has been directed to find suitable water based systems for film coating in drug delivery systems.

Latex particles in water as the dispersion medium have been known for almost half a century. These particles are polymeric colloidal particles in the 10 to 100 nm range and have been utilized as film formers in different applications. If the polymer particle has a sufficiently low glass transition temperature (Tg) when the water is evaporated, the particles can coalesce to form a film. Further development has given several other products that have been tested and reported in a number of publications (Petereit and Weisbrod, Eur J Pharmaceutics and Biopharm 47, 15 (1999); Petereit et al, Ibid, 41, 219 (1995); Amighi and Moës, STP Pharma Sci 7, 141 (1997); Bodmeier and Paeratukul, Pharm Res 11, 882 (1994); Ozturk et al, J Controlled Release 14, 203 (1990). Goodhart et al, Pharmaceutical Tech April, 64 (1984); Bodmeier and Paeratakul Int J Pharmceutics 152, 17 (1997); Bodmeier and Paeratakul Drug Develop Ind Pharmacy 20, 1517 (1994)).

From these and other studies it can be concluded that one of the more interesting dispersions, due to the low Tg of the latex polymer and high elongation, is Eudragit® NE 30D, which contains approximately 28.5% w/w particles of the copolymer poly(ethylacrylate-co-methylmethacrylate), and 1.5% w/w of the non-ionic tenside Nonoxynol 100 (a polyoxyethylated nonylphenol) as a stabiliser. The advantages of this copolymer are that no plasticizer is needed for film formation, the copolymer has a low film forming temperature and the high flexibility of dried films. The last property is especially useful when preparing tablets. However, to obtain best spraying conditions and technical performance, an anti-sticking agent has to be added to the dispersion, as reported by Petereit and Weisbrod 1995, due to the tacky property of the pellets during coating. Several such agents are available e.g., talc, silica, magnesium stearate and glyceryl mono stearate (GMS). It was reported, however, that best performance of the dispersion during spraying and of the dried film was obtained when the GMS was dispersed with an extra surface active agent, e.g., Polysorbate 80 (PS80). The reason for adding PS80 is to be able to make a stabile dispersion between GMS and PS80 and to make a dispersion that has a small particle size distribution.

We have found that it has been difficult to obtain results with acceptable reproducibility with respect to, e.g., release rates and stability during storage from formulations manufactured according to these suggested procedures. It is important that the release profile is stable during the shelf life of the product and also stable in different enviroments.

Instabilities of coated dosage (water based) forms are mainly based on physical interaction caused, most likely, by improper formulations of coating suspension (i.e., plasticizers, surfactants, or pigments), or the film coating process (Petereit et al, Eur. J pharmaceutics and Biopharmaceutics 47, (1999) 15-25). Residual moisture, probably enclosed in the core and, migrating over time, may increase the permeability of coatings due to plasticizing effects. This increased permeability can also be due to migration of components in the film coat during storage in different climates, thus resulting in a coating which exhibits altered release properties
Eur J Pharm Biopharm 41 (4) (1995) 219-228 by Petereit et al discloses the use of GMS as a glidant in aqueous film coating formulations comprising Eudragit® L 30 D-55, NE 30D, RL 30D and RS 30D.
Eur J Pharm Biopharm 47 (1999) 73-78 by Wesseling et al discusses how reduced tackiness of NE30D films is obtained by the addition of an anti-tacking agent, e g GMS. This is achieved by the addition of GMS in an emulsion form that is obtained by mixing a surfactant (e g Tween 80) with the GMS.
Eur J Pharm Biopharm 1999, 14 (6) p743-751 by Petereit, Weisbrod is a review article that gives compositions of dispersions for film formation in tables. However, nothing is said about how the additives, e g anti-tacking agents, are added.
AAPS Pharmasci 2001 3(2) 1-11 by Lin et al discloses that the endogenous surfactant of NE30D, nonoxynol 100, crystallizes in films of NE30D. The importance of compensating for this tendency for crystallization is discussed.
J Microencapsulation 1997, vol 14, no 6, p743-751 by Mathir et al discloses coatings comprising NE30D, talc and PEG 6000.
U.S. Pat. No. 5,817,776 discloses that a dispersion of lubricant/glidant is formed in water with the help of an “antifoam suspension”.
U.S. Pat. No. 5,478,573 discloses that mixtures of different dispersions are prepared and stabilized with the surface active agent sodium lauryl sulfate. Also disclosed is a coating comprising magnesium stearate dispersed in water and added to a mixture of the two dispersions NE30D and Aquacoat™.
U.S. Pat. No. 4,784,858 discloses that the anti-tacking agent talc is added together with a surfactant (Tween 80) to Eudragit E30D.
WO 02058677 discloses coating formulations comprising sodium stearyl fumarate (PRUV®).

### PURPOSE OF THE INVENTION

The purpose of the present invention is to provide a new film coating system that does not have the above mentioned problems. Improved properties of the new film coating system are, for example, non-stickiness, high mechanical strength, reproducibility during processing, and stability of the film coating during storage.

### SUMMARY OF THE INVENTION

We have surprisingly found a novel film coating composition which provides a latex dispersion suitable for coating pharmaceutical formulations wherein the film produced serves as a barrier giving close to constant release (zero-order) from the formulation. In addition, the physical properties of the film produced no processing problems, such as aggregation of particles; the film exhibited high mechanical strength; and the film was stable exhibiting no change in release properties as a result of storage. Moreover, the film could be reproducibly made having these improved properties.

Accordingly, the invention features a film coating composition, a film coating, a formulation including a pharmaceutically core comprising a pharmaceutically active agent (e.g., metoprolol) and a film coat. The invention further includes a process of making the coating and a process to prepare a formulation having the coating of the invention.

### DETAILED DESCRIPTION OF THE INVENTION

The present invention provides a film coating which, has the unexpected property of having a stable release profile following storage. Specifically, the present invention features a pharmaceutical composition which surprisingly has a low amount of surface active agent.

Accordingly, the invention provides a film coating composition suitable for use in coating pharmaceutical formulations comprising a dispersion which comprises:
a) an acrylic polymer, which is Eudragit® NE30D
b) an anti-sticking agent, which is glyceryl monostearate (GMS)
c) a surface active agent wherein the surface active agent is in an amount less than 1.3% by weight of the dispersion, and
d) a water-containing liquid,
   wherein the dispersion does not contain a vinyl acetate polymer.

In another aspect, the invention provides a film coat covering a pharmaceutical core wherein the core includes a pharmacologically active ingredient and optionally one or more pharmaceutically acceptable excipients. The film coat includes an:
a) an acrylic polymer, which is Eudragit® NE30D
b) an anti-sticking agent; which is glyceryl monostearate (GMS) and
c) a surface active agent wherein the surface active agent is in the amount less than 5.4% by weight of the weight of the film coat, and wherein the film coat has been deposited from a water-containing liquid and does not contain a vinyl acetate polymer. In one embodiment, the acrylic polymer can be an ethyl acrylate and/or methyl methacrylate copolymer, the anti-sticking agent can be glyceryl mono stearate (GMS), and the surface active agent can be nonoxynol 100. Suitably the film coat has a thickness in the range of 1 to 100 micrometres, preferably in the range of 5 to 50 micrometres and more preferably in the range of 10 to 30 micrometres

The pharmacologically active ingredient can be provided in a plurality of beads, optionally containing one or more pharmaceutically acceptable excipients, wherein each of the beads is coated with a film coat as defined above. Such film coated beads may be provided in sachets or formulated as a capsule, for example a hard gelatin capsule, or compressed to form tablets using known methods with the optional addition of other pharmaceutically acceptable additives. Coated beads to be compressed into a tablet are obtained by conventional techniques known to those skilled in the art. Also, during this process suitable other agents can be added. For example, during the tabletting step suitable fillers, e.g., microcrystalline cellulose, talc, sodium stearyl fumarate, etc., can be utilised to give acceptable compression characteristics of the formulation, e.g., hardness of the tablet.

Optionally the beads may contain an insoluble core onto which the active ingredient has been deposited, for example, by spraying. Suitable materials for the inert core are silicon dioxide, glass or plastic resin particles. Suitable types of plastic material are pharmaceutically acceptable plastics such as polypropylene or polyethylene preferably polypropylene. Such insoluble cores have a size diameter in the range of 0.01-2 mm, preferably in the range of 0.05-0.5 mm and more preferably in the range of 0.1-0.3 mm.

In one embodiment, the ductility of the film can be in a range of 500-20000 J/m³, In another embodiment the ductility is in the range of 2500-20000 J/m³. In yet another embodiment the ductility is in the range of 10000-20000 J/m³.

In another aspect, the invention provides a pharmaceutical formulation which includes
a) a pharmaceutical core comprising a pharmacologically active ingredient and optionally one or more pharmaceutically acceptable excipients, and
b) a film coat comprising:
   i) an acrylic polymer, which is Eudragit® NE30D
   ii) an anti-sticking agent, which is glyceryl monostearate (GMS)
   iii) a surface active agent wherein the surface active agent is in the amount less than 5.4% by weight of the weight of the film coat,
      wherein the film coat has been deposited from a water-containing liquid and does not contain a vinyl acetate polymer.

The pharmacologically active ingredient can be provided in a plurality of beads, optionally containing one or more pharmaceutically acceptable excipients, wherein each of the beads is coated with a film coat as defined above. Such film coated beads may be provided in sachets or formulated as a capsule, for example a hard gelatin capsule, or compressed to form tablets using known methods with the optional addition of other pharmaceutically acceptable additives. Coated beads to be compressed into a tablet are obtained by conventional techniques known to those skilled in the art. Also, during this process suitable other agents can be added. For example, during the tabletting step suitable fillers, e.g., microcrystalline cellulose, talc, etc., can be utilised to give acceptable compression characteristics of the formulation, e.g., hardness of the tablet. Suitably the beads have a diameter in the range of 0.01-2 mm, preferably in the range of 0.05-11.0 mm and more preferably in the range of 0.1-0.7 mm.

Optionally the beads may contain an insoluble core onto which the active ingredient has been deposited, for example, by spraying. Suitable materials for the inert core are silicon dioxide, glass or plastic resin particles. Suitable types of plastic material are pharmaceutically acceptable plastics such as polypropylene or polyethylene preferably polypropylene. Such insoluble cores have a size diameter in the range of 0.01-2 mm, preferably in the range of 0.050.5 mm and more preferably in the range of 0.1-0.3 mm.

In one embodiment, the ductility of the film can be in a range of 500-20000 J/m³ In another embodiment the ductility is in the range of 2500-20000 J/m³. In yet another embodiment the ductility is in the range of 10000-20000 J/m³.

In a more preferred aspect the present invention provides a modified release formulation wherein the pharmacologically active ingredient is released over a long period of time, for example longer than 3 hours in comparison to an immediate release tablet e.g., up to 24 hours, in comparison to an immediate release tablet Preferably the pharmacologically active ingredient is released from the formulation over 10 to 24 hours, for example over 18 to 22 hours.

Preferably the pharmacologically active ingredient has activity in the treatment of cardiovascular diseases. In particular, the pharmacologically active ingredient is a beta-blocking adrenergic agent such as metoprolol or a pharmaceutically acceptable salt thereof.

In yet another aspect the invention provides a modified release metoprolol formulation including:
a) a metoprolol core comprising metoprolol or a pharmaceutically acceptable salt thereof and optionally one or more pharmaceutically acceptable excipients; and
b) a film coat as defined above.

In a preferred aspect the core comprising metoprolol or a pharmaceutically acceptable salt thereof includes a plurality of beads which comprise metoprolol or a pharmaceutically acceptable salt thereof and optionally one or more pharmaceutically acceptable excipients wherein each of the beads is coated with a film-coat as defined above. Preferably, the beads have an inert core as described previously.

Suitable pharmaceutically acceptable salts of metoprolol include the tartrate, succinate, fumarate or benzoate salts and especially the succinate salt. The S-enantiomer of metoprolol or a salt thereof, particularly the benzoate salt or the sorbate salt, may also be used.

Eudragit® NE30D is an ethyl acrylate/methyl methacrylate copolymer in which the ethyl acrylate concentration is about 67 mol %.

It was surprisingly found that the film coating of the present invention could be made with low amounts of surface active agents and that such a coating exhibited stability over time. The surface active agent (surfactant) can act as a stabilizer.

Suitably the amount of surface active agent used in a film coating dispersion is in the amount less than 1.3%, e.g., 1.0, 0.9, 0.8, 0.7, 0.6, 0.5, 0.4, 0.3, 0.2, or 0.1%, by weight of the dispersion. In one embodiment, the surface active agent is in a range from 0.001-1.0% by weight of the dispersion. In another embodiment, the surface active agent is in a range from 0.01-0.8% by weight of the dispersion. In yet another embodiment, the surface active agent is in a range from 0.1-0.5% by weight of the dispersion.

Suitably the amount of surface active agent present in the film coat is less than 5.4% by weight of the weight of the film coat, e.g., less than 5.0, 4.5, 3.5, 3.0, 2.5, 2.0, 1.5, 1.0, 0.5, 0.1, 0.01, or 0.001% by weight of the weight of the film coat. In one embodiment, the surface active agent in the film coat is in a range from 0.01-5.0% by weight of the weight of the film coat. In another embodiment, the surface active agent is in a range from 0.1-4.0% by weight of the weight of the film coat. In yet another embodiment, the surface active agent is in a range from 1.0-3.0% by weight of the weight of the film coat

Suitably the amount of surface active agent present in the dispersion is less than 5.6%, e.g., less than 5.0, 4.5, 3.5, 3.0, 2.5, 2.0, 1.5, 1.0, 0.5, 0.1, 0.01, or 0.001%, based on the dry weight of the film coating components.

Examples of suitable surface active agents\stabilizers include:
a nonionic surfactant, like sorbitan esters (Span series); polysorbates (Tween series);
polyoxyethylated glycol monoethers (like the Brij series); polyoxyethylated alkyl phenols (like the Triton series or the Igepal series); alkyl glucosides (e.g., dodecylmaltoside); sugar fatty acid esters (e.g., sucrose laurate); saponins; etc: or mixtures thereof;
ampholytic surfactants, like betaines;
anionic surfactants, like sulphated fatty alcohols eg sodium dodecylsulphate SDS; sulphated polyoxyethylated alcohols; others like dioctyl sulphosuccinate; bile salts (e.g., dihydroxy bile salts like sodium deoxycholate, trihydroxy bile salts like sodium glycocbolate, etc); fusidates (e.g., sodium dihydrofusidate); etc cationic surfactants, like ammonium compounds;
soaps, fatty acids, and lipids and their salts, like alkanoic acids; (e.g., octanoic acid, oleic acid); monoglycerides (eg monolein), phospholipids which are neutral or positively or negatively charged (eg dialkyl phosphatidylcholine, dialkyl phosphatidylserine, etc).

More preferably the surface active agent is a nonionic surfactant. Most preferably the surface active agent is nonoxynol 100.

Optionally, other different excipients can be included in the formulation by methods known to those skilled in the art, such as lubricants, plasticizers, etc.

Suitably the water-containing liquid comprises water and a water miscible organic liquid for example lower alkanols e.g. ethanol, propanol or isopropanol. From a safety point of view it is preferred that the proportion of the organic is kept to a minimum but small amounts are tolerable for example in the range of 0 to 20% by volume. Preferably the liquid is water.

The film-coating composition is particularly suitable for use as an aqueous film-coating composition wherein the film-coat is applied using water as the liquid. This process is particularly advantageous as it negates the need to use environmentally unacceptable organic solvents, some of which also present processing problems due to their inflammablility, while also eliminating many of the problems experienced with aqueous coatings described above.

In another aspect the present invention provides processes for the preparation of the film-coating composition. Therefore, there is provided a process for the preparation of a film-coating composition comprising mixing together the acrylic polymer dispersion, vinyl acetate polymer dispersion, the stabilizer and the liquid at a temperature in the range of 10 to 100° C.

In one embodiment of the process the acrylic polymer dispersion, the stabilizer, the surface active agent and the liquid, are mixed at room temperature.

Suitably mixing is achieved by methods such as stirring or shaking but other methods of homogenization known to those skilled in the art may be used.

In another aspect the present invention provides a process for film coating a pharmaceutical core wherein a film coating composition as defined above is applied to a core. Preferably the film coating composition is applied by spraying for example in a fluidised bed with top spray or bottom spray techniques. Other coating methods used are coating in standard coating pans with perforated pans, Accela-cota, immersion swords, Glatt, or immersion tubes as described in “Theory and Practice in Industrial Pharmacy” edited by Lachman, published by Lea and Feabiger 1986 3^{rd} edition.

In another aspect the invention provides a process to prepare a film coat as defined above comprising removing the liquid from a film coating composition as defined above. Suitably the liquid is removed by evaporation for example by spray drying for example in a fluidised bed.

In yet another aspect the invention provides a process to prepare a formulation as defined above comprising coating a pharmaceutical core as defined above with a film coating composition as defined above.

In a further aspect the invention provides a process to prepare a formulation in which the pharmacologically active ingredient is provided as a plurality of beads as defined above comprising coating the plurality of beads with a film-coating composition as defined above.

### EXAMPLES

The following examples are non-limiting and are given by way of illustration only. It will be appreciated by those skilled in the art that the examples are to be looked upon as guidelines, and the invention is not restricted to the exemplified compositions. A wide range of combinations is possible to give film coatings having the necessary properties required for each specific application.

### Example 1

Preparation of Coated Metoprolol Succinate Pellets From NE30D/GMS/PS80. The Preparation Contains:

5.0 GMS+190 g water+1 g Polysorbate80+25 g cold water+334.6 g NE 30D
(giving GMS/particle ratio approximately 5% and PS80/GMS ratio approximately 20%)

The mixture of 5 g GMS, 1 g Polysorbate 80 and 190 g water was heated to 60-63° C. while stifling. After 30 minutes (min) of homogenizing, the mixture was cooled initially by adding 25 g cold water and then further cooled in a cold-water bath until the temperature was below 35° C. The solution was then slowly poured into the NE30D dispersion while gently stirring. Stirring was continued until the start of coating.

The coating was performed in a small-scale coater. Coating was performed by pouring 400 g of metoprolol succinate pellets (size fraction 0.400.63 mm, with inert silicon dioxide cores) into the coater and the solution sprayed on to it. The coated pellets where cured afterwards in a 60° C. heat cabinet for one (1) hour.

The coating yield was 92%. The drug content for the pellets after coating: 630 mg/g pellets. The coated pellets where then analysed regarding drug content and release profile. Dissolution tests were conducted in USP 23 paddle apparatus equipped with flow-through cells (at 37° C., 100 rpm, 500 ml sodium phosphate buffer solution, pH 6.8). The release from the coated pellets and tablets was measured online using an UV-spectrophotometer (Lambda 2, Perkin-Elmer) at 274 nm The amount of drug release was calculated on the determined drug content.

TABLE 1

| | | 7 days | 7 days | 7 days | 14 days | 14 days | 14 days |
|---|---|---|---|---|---|---|---|
| Release | Zero | in cli- | in cli- | in cli- | in cli- | in cli- | in cli- |
| time (h) | value | mate 1 | mate 2 | mate 3 | mate 1 | mate 2 | mate 3 |
| 1 | 0.3 | 0.3 | 0.4 | 0.5 | 0.3 | 0.3 | 0.4 |
| 2 | 0.4 | 0.3 | 0.4 | 0.6 | 0.3 | 0.3 | 0.5 |
| 3 | 0.4 | 0.4 | 0.5 | 0.7 | 0.4 | 0.4 | 0.6 |
| 4 | 0.4 | 0.4 | 0.5 | 0.87 | 0.4 | 0.5 | 0.9 |
| 6 | 0.5 | 0.6 | 0.8 | 2.4 | 0.5 | 0.8 | 4.1 |
| 8 | 0.6 | 1.0 | 1.7 | 6.9 | 0.9 | 2.3 | 11.4 |
| 10 | 1.2 | 2.1 | 4.4 | 13.5 | 2.3 | 6.9 | 20.6 |
| 12 | 2.6 | 5.9 | 13.6 | 21.4 | 7.7 | 19.6 | 30.5 |
| 14 | 8.2 | 19.8 | 35.4 | 30.1 | 26.6 | 42.4 | 40.8 |
| 16 | 23.6 | 44.7 | 58.4 | 39.6 | 52.9 | 62.3 | 51.1 |
| 18 | 44.3 | 64.8 | 72.7 | 49.4 | 70.4 | 75.1 | 60.9 |
| 20 | 61.1 | 76.2 | 81.2 | 58.6 | 80.1 | 82.7 | 69.2 |

Zero value: release value after curing (60° C./50% relatively humidity (r.h.) during 60 min).
Climate 1; 25° C./60% r.h.
Climate 2; 30° C./60% r.h.
Climate 3; 40° C./75% r.h.
Each value is a mean value of two analyses.

Results

The results show how the release profiles change with time and storage conditions. Samples stored in higher temperatures and humidity tended to have a more linear profile compared to those samples in lower temperatures and humidity. This change during storage is most likely due to migration of surfactants, especially polysorbate 80, up to surface leading to increased permeability.

### Example 2

Preparation of Coated Metoprolol Succinate Pellets From NE30D/GMS Solution. The Preparation Contains:

3.0 g GMS+150 g water+25 g cold water+200 g NE 30D
(giving GMS/particle ratio approximately 5% and film amount/pellets ratio approximately 20%)

The mixture of 3 g GMS and 150 g water was heated to 60-63° C. while stirring. After 30 minutes the mixture was cooled initially by adding 25 g cold water and then further cooled in a cold-water bath until the temperature was below 35° C. The solution was then slowly poured into the NE30D dispersion while gently stirring. Stirring was continued until the start of coating.

The coating was performed in a small-scale coater. Coating was performed by pouring 350 g of metoprolol succinate pellets (size fraction 0.40-0.63 mm, with inert silicon dioxide cores) into the coater and the solution sprayed on to it. The pellets where cured afterwards in a 60° C. heat cabinet for 6 min. The coating yield was 90%. The drug content for the pellets after coating: 659 mg/g pellets.

The coated pellets were then stored in a climate cabin at different temperatures and humidity for one and two weeks. The coated pellets where then analysed regarding drug content and release profile.

Dissolution tests were conducted in USP 23 paddle apparatus equipped with flow-through cells (at 37° C., 100 rpm, 500 ml sodium phosphate buffer solution, pH 6.8). The release from the coated pellets and tablets was measured online using an UV-spectrophotometer Lambda 2, Perkin-Elmer) at 274 nm. The amount of drug release was calculated on the determined drug content.

Results

The release profile was analysed for the coated pellets after final drying (curing) and after 7 and 14 days in three different climates, see table 2.

TABLE 2

| Release | | 7 days | 7 days | 7 days | 14 days | 14 days | 14 days |
|---|---|---|---|---|---|---|---|
| Time | Zero | in cli- | in cli- | in cli- | in cli- | in cli- | in cli- |
| (h) | value | mate 1 | mate 2 | mate 3 | mate 1 | mate 2 | mate 3 |
| 1 | 0.49 | 0.3 | 0.3 | 0.2 | 0.3 | 0.2 | 0.3 |
| 2 | 1.6 | 0.76 | 0.5 | 0.3 | 0.68 | 0.3 | 0.6 |
| 3 | 3.7 | 1.9 | 1.1 | 0.4 | 1.4 | 0.7 | 0.8 |
| 4 | 6.98 | 3.9 | 2.3 | 0.7 | 2.8 | 1.6 | 1.2 |
| 6 | 17.4 | 13.3 | 10.4 | 3.5 | 10.9 | 9.4 | 3.7 |
| 8 | 27.9 | 25.5 | 24.8 | 13.85 | 24.1 | 25 | 13 |
| 10 | 37.6 | 36.1 | 37.2 | 27.6 | 35.4 | 38.3 | 25.8 |
| 12 | 47.2 | 46.1 | 48.3 | 40.5 | 45.8 | 49.9 | 38.6 |
| 14 | 56.5 | 55.7 | 58.5 | 52.35 | 55.6 | 60.4 | 50.4 |
| 16 | 65.1 | 64.55 | 67.3 | 62.2 | 64.6 | 69.1 | 60.4 |
| 18 | 72.5 | 72.4 | 74.6 | 69.8 | 72.1 | 75.7 | 68.1 |
| 20 | 78.3 | 77.8 | 80.2 | 75.4 | 78.1 | 80.8 | 74 |

Climate 1; 25° C./60% r.h.
Climate 2; 30° C./60% r.h.
Climate 3; 40° C./75% r.h.
Each value is a mean value of two analysis.

Results

The results shows similar profiles after 7 and 14 days in the three different climates. No migration can be seen in Example 2.

### Example 3

### Tablets Made From the Coated Pellets of Example 2

Preparation of 950 Tablets

The coated pellets where compressed into tablets by conventional techniques. During this process other agents where added, fillers Avicel and Sodium stearyl fumarate (Pruv®) to give acceptable compression characteristics of the formulation, e g hardness and weight.

136.9 g of coated pellets (from experiment 2) were mixed with 205.3 g of microcrystalline cellulose (Avicel PH 102 (course), FMC) in a Turbula mixer (W. A. Bachofen, Switzerland) for 4 minutes. After this time 0.68 g Sodium stearyl fumarate was added and mixing was continued for another minute. The tablet weight was calculated to be 361 mg.

Tablets of pellets were compacted in a rotary tablet machine (six (6) punch press, Korch PH 106, Germany). The press was equipped with concave-faced punches with a diameter of 10 mm. The force used was approximately 4 KN. The hardness of the tablets were approx. 142 N (mean value of ten measurements). After the tablets have been sorted out by weight, they were put in the same cabin as the coated pellets from Example 2, and samples were taken after one and four weeks. See Table 3.

The release profile of the tablets was then analysed.

Dissolution tests were conducted in USP 23 paddle apparatus equipped with flow-through cells (at 37° C., 100 rpm, 500 ml sodium phosphate buffer solution, pH 6.8). The release from the coated pellets and tablets was measured online using an UV-spectrophotometer (Lambda 2, Perkin-Elmer) at 274 nm. The amount of drug release was calculated on the determined drug content.

TABLE 3

| Release | | 7 days | 7 days | 7 days | 28 days | 28 days | 28 days |
|---|---|---|---|---|---|---|---|
| Time | Zero | in cli- | in cli- | in cli- | in cli- | in cli- | in cli- |
| (h) | value | mate 1 | mate 2 | mate 3 | mate 1 | mate 2 | mate 3 |
| 1 | 15 | 19 | 19 | 13 | 19 | 18 | 17 |
| 2 | 27 | 34 | 34 | 29 | 35 | 32 | 32 |
| 3 | 37 | 44 | 43 | 41 | 45 | 42 | 43 |
| 4 | 45 | 52 | 51 | 49 | 52 | 50 | 51 |
| 6 | 58 | 62 | 61 | 60 | 63 | 61 | 62 |
| 8 | 67 | 70 | 69 | 68 | 71 | 70 | 69 |
| 10 | 74 | 77 | 75 | 74 | 77 | 76 | 75 |
| 12 | 79 | 82 | 80 | 79 | 83 | 82 | 79 |
| 14 | 84 | 87 | 85 | 84 | 87 | 87 | 83 |
| 16 | 87 | 91 | 89 | 87 | 91 | 90 | 86 |
| 18 | 90 | 94 | 92 | 90 | 93 | 93 | 89 |
| 20 | 93 | 97 | 94 | 93 | 95 | 96 | 91 |

Each value is a mean value of two analysis.
Climate 1; 25° C./60% r.h.
Climate 2; 30° C./60% r.h.
Climate 3; 40° C./75% r.h.

Results

The results showed no major change in the release profile during storage in the different climates.

1. A film coating composition suitable for use in coating pharmaceutical formulations, wherein the composition comprises a dispersion comprising:
a) an ethyl acrylate/methyl methacrylate copolymer;
b) an anti-sticking agent, which is glyceryl monostearate (GMS);
c) a surface active agents wherein the surface active agent is present in an amount less than 1.3% by weight of the dispersion; and
d) a water-containing liquid,
wherein the dispersion does not contain a vinyl acetate polymer.

2. A film coat covering a pharmaceutical core, wherein the core comprises a pharmacologically active ingredient and optionally one or more pharmaceutically acceptable excipients, and wherein the film coat comprises:
a) an ethyl acrylate/methyl methacrylate copolymer;
b) an anti-sticking agent, which is glyceryl monostearate (GMS); and
c) a surface active agent, wherein the surface active agent is present in an amount of less than 5.4% by weight of the weight of the film coat, and
wherein the film coat has been deposited on the pharmaceutical core from a water-containing liquid and does not contain a vinyl acetate polymer.

3. A pharmaceutical formulation comprising:
a) a pharmaceutical core comprising a pharmacologically active ingredient and optionally one or more pharmaceutically acceptable excipients, and
b) a film coat covering the pharmaceutical core, wherein the film coat comprises:
i) an ethyl acrylate/methyl methacrylate copolymer;
ii) an anti-sticking agent, which is glyceryl monostearate (GMS); and
iii) a surface active agent, wherein the surface active agent is present in an amount of less than 5.4% by weight of the weight of the film coat, and
wherein the film coat has been deposited on the pharmaceutical core from a water-containing liquid and does not contain a vinyl acetate polymer.

4. A pharmaceutical formulation comprising a plurality of beads containing a pharmacologically active ingredient and optionally one or more pharmaceutically acceptable excipients, wherein each of the beads is coated with a film coat comprising:
a) an ethyl acrylate/methyl methacrylate copolymer;
b) an anti-sticking agent, which is glyceryl monostearate (GMS); and
c) a surface active agent, wherein the surface active agent is present in an amount of less than 5.4% by weight of the weight of the film coat, and
wherein the film coat has been deposited on the beads from a water-containing liquid and does not contain a vinyl acetate polymer.

5. The formulation according to claim 3 or 4, wherein the formulation is a modified release formulation.

6. The formulation according to claim 5, wherein the pharmacologically active ingredient has activity in the treatment of cardiovascular diseases.

7. The formulation according to claim 6, wherein the pharmacologically active ingredient is a beta-blocking adrenergic agent.

8. The formulation according to claim 7, wherein the pharmacologically active ingredient is metoprolol or a pharmaceutically acceptable salt thereof.

9. The formulation according to claim 8, wherein the metoprolol salt is a tartrate, succinate, fumarate, or benzoate salt.

10. The composition as claimed in claim 1, wherein the liquid is water.

11. A process for the preparation of a film coating composition according to claim 1, the process comprising mixing together the ethyl acrylate/methyl methacrylate copolymer, the anti-sticking agent, the surface active agent, and the liquid at a temperature in the range of 10 to 100° C. to form a dispersion.

12. A process for the preparation of a pharmaceutical formulation as claimed in claim 3, comprising coating the pharmaceutical core with a film coating composition, wherein the composition comprises a dispersion comprising:
a) an ethyl acrylate/methyl methacrylate copolymer;
b) an anti-sticking agent, which is glyceryl monostearate (GMS);
c) a surface active agent wherein the surface active agent is present in an amount less than 1.3% by weight of the dispersion; and
d) a water-containing liquid, and
wherein the dispersion does not contain a vinyl acetate polymer.

13. A process for the preparation of a pharmaceutical formulation according to claim 4, the process comprising coating each of the plurality of beads with a film coating composition, wherein the composition comprises a dispersion comprising:
a) an ethyl acrylate/methyl methacrylate copolymer;
b) an anti-sticking agent, which is glyceryl monostearate (GMS);
c) a surface active agent wherein the surface active agent is present in an amount less than 1.3% by weight of the dispersion; and
d) a water-containing liquid, and
wherein the dispersion does not contain a vinyl acetate polymer.

14. The film coating composition according to claim 1, wherein the ethyl acrylate/methyl methacrylate copolymer is Eudragit® NE30D.

15. The film coat according to claim 2, wherein the ethyl acrylate/methyl methacrylate copolymer is Eudragit® NE30D.

16. The pharmaceutical composition according to claim 3, wherein the ethyl acrylate/methyl methacrylate copolymer is Eudragit® NE30D.
